(19) United States
(12) Patent Application Publication
(10) Pub. No.: US 2016/0263301 A1
(43) Pub. Date: Sep. 15, 2016

(54) Pulmonary Arterial Hypertension Treatment Devices and Related Systems and Methods

(71) Applicant: Sunshine Heart Company Pty, Ltd., Clontarf (AU)

(72) Inventors: David Rosa, Eden Prairie, MN (US); Dimitrios Georgakopoulos, Plymouth, MN (US); Martin C. Cook, Eden Prairie, MN (US); Tolga Tas, Oxford (GB)

(21) Appl. No.: 15/065,650

(22) Filed: Mar. 9, 2016

Related U.S. Application Data
(60) Provisional application No. 62/130,138, filed on Mar. 9, 2015.

(51) Int. Cl. A61M 1/12

(52) U.S. Cl. CPC A61M 1/122; A61M 1/125; A61M 1/127; A61M 2205/04; A61M 2210/12

(57) ABSTRACT

Systems, methods, and devices for treating pulmonary arterial hypertension are provided. The system comprises an implantable actuator that compresses the pulmonary artery.

### CROSS-REFERENCE TO RELATION APPLICATION(S)

This application claims the benefit under 35 U.S.C. §119(e) to U.S. Provisional Application 62/130,138, filed Mar. 9, 2015 and entitled “Pulmonary Arterial Hypertension Treatment Devices and Related Systems and Methods,” which is hereby incorporated herein by reference in its entirety.

### FIELD OF THE INVENTION

The embodiments herein relate to various devices for treating pulmonary arterial hypertension and related systems and methods. Exemplary devices including actuators configured to stress the pulmonary artery.

### BACKGROUND OF THE INVENTION

Pulmonary arterial hypertension (“PAH”) is a disease in which the pressures in the pulmonary artery exceed 25 mmHg. This can be associated with increased pulmonary vascular resistance (“PVR”), a reduction in pulmonary compliance or elevated filling pressure of the left ventricle Elevated pressures in the pulmonary artery contribute to the syndrome of heart failure and PAH by increasing global and renal sympathetic activity, leading to systemic vasoconstriction and water and salt retention.

Inhalation of nitrous oxide (“NO”) is a common treatment for PAH management. In addition, there are several pharmacological supports that aid in lowering the PVR by creating a vasodilation effect. One disadvantage of these existing therapies is that they are applied generally to the entire body and thus affect the entire body, thereby resulting in some side effects.

There is a need in the art for improved methods, systems, and devices to facilitate localized pulmonary endothelial nitric oxide release.

### BRIEF SUMMARY OF THE INVENTION

Discussed herein are various arterial compression devices and related systems and methods for treating pulmonary arterial hypertension. The compression devices in certain implementations are configured to cause the pulmonary artery to release nitric oxide. In other embodiments, the devices are configured to cause improved filling of the patient's left ventricle.

In Example 1, a heart assist system comprises an arterial compression device configured to be positioned adjacent to a pulmonary artery of a patient, a pump in fluid communication with the arterial compression device, and a power source operably coupled to the pump. The arterial compression device is configured to compress the pulmonary artery, whereby the arterial compression device is configured to cause the pulmonary artery to release nitric oxide. The pump is configured to pump a fluid to the arterial compression device so as to actuate the arterial compression device. The power source comprises a battery or a transcutaneous electronic transfer device.

In Example 2, a heart assist system comprises an arterial compression device configured to be positioned adjacent to a pulmonary artery of a patient, a pump in fluid communication with the arterial compression device, and a power source operably coupled with the pump. The arterial compression device is configured to compress the pulmonary artery, whereby the arterial compression device is configured to cause improved filling of a left ventricle of the patient. The pump is configured to pump a fluid to the arterial compression device so as to actuate the arterial compression device. The power source comprises a battery or a transcutaneous electronic transfer device.

Example 3 relates to the heart assist system according to Example 2, wherein the arterial compression device is configured to cause improved filling of the left ventricle by increasing forward flow into the left ventricle and reducing afterload in a right ventricle.

In Example 4, a method of treating pulmonary arterial hypertension comprises positioning an arterial compression device adjacent to a pulmonary artery of a patient, and compressing the pulmonary artery with the arterial compression device, whereby the arterial compression device is configured to cause the pulmonary artery to release nitric oxide.

In Example 5, a method of treating pulmonary arterial hypertension comprises positioning an arterial compression device adjacent to a pulmonary artery of a patient, and causing the pulmonary artery to release nitric oxide by compressing the pulmonary artery with the arterial compression device.

In Example 6, a method of treating pulmonary arterial hypertension comprises positioning an arterial compression device adjacent to a pulmonary artery of a patient, and compressing the pulmonary artery with the arterial compression device, whereby the arterial compression device is configured to cause improved filling of a left ventricle of the patient.

Example 7 relates to the method according to Example 6, wherein the arterial compression device is configured to cause improved filling of the left ventricle by increasing forward flow into the left ventricle and reducing afterload in a right ventricle.

In Example 8, a method of treating pulmonary arterial hypertension comprises positioning an arterial compression device adjacent to a pulmonary artery of a patient, and causing improved filling of a left ventricle of the patient by compressing the pulmonary artery with the arterial compression device.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a schematic drawing of a first embodiment of a pulmonary arterial hypertension treatment system implanted in the thoracic cavity of a patent, according to one embodiment.

FIG. 2 is a front view of an actuator wrap, according to one embodiment.

FIG. 3A is a cross-sectional, cutaway view of an actuator, according to one embodiment.

FIG. 3B is a cross-sectional, cutaway view of the actuator of FIG. 3A positioned against a pulmonary artery.

FIG. 4 is a cross-sectional, cutaway view of an actuator, according to a further embodiment.

FIG. 5A is an exploded perspective view of an actuator, according to one embodiment.

FIG. 5B is a cross-sectional, cutaway view of the actuator of FIG. 5A.

FIG. 6A is an cross-sectional, cutaway view of an actuator, according to one embodiment.

FIG. 6B is a cross-sectional, cutaway view of the actuator of FIG. 6A.

### DETAILED DESCRIPTION

The various embodiments disclosed or contemplated herein relate to actuator devices for compressing or otherwise externally, physically stressing the pulmonary artery and related systems and methods. The compression of the pulmonary artery causes shear stress in the endothelial tissue, which causes the release of endogenous nitric oxide, which helps to treat PAH. In certain implementations, the compression is a counterpulsation of the PA, which can reduce the afterload on the right ventricle of the heart. Further, the counterpulsation of the PA also serves to improve filling of the left ventricle, due to the influence of the counterpulsation on the left atrium.

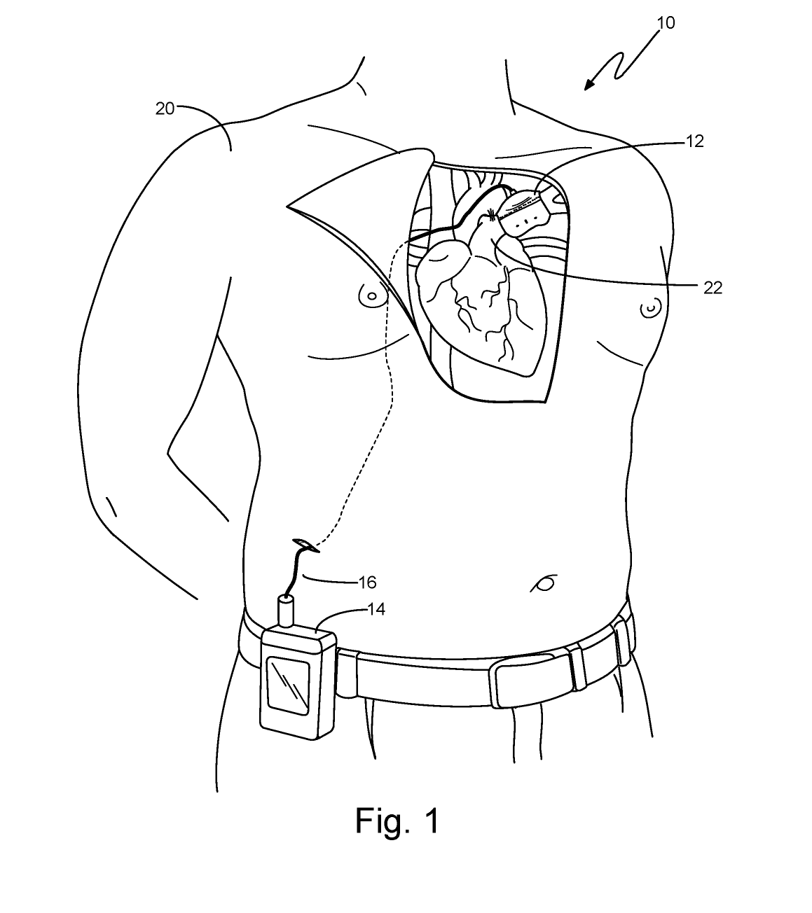

FIG. 1 is a schematic drawing showing one embodiment of a PAH treatment system 10. The system 10 has an actuator 12 that is suitable for complete implantation in the thoracic cavity of a subject 20 adjacent the pulmonary artery 22, as shown.

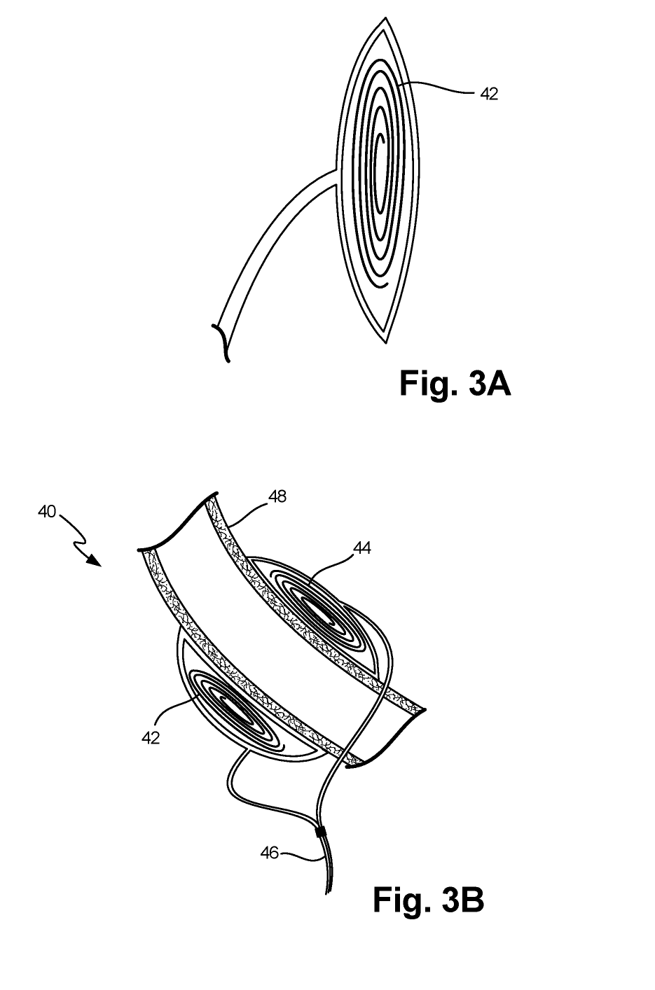

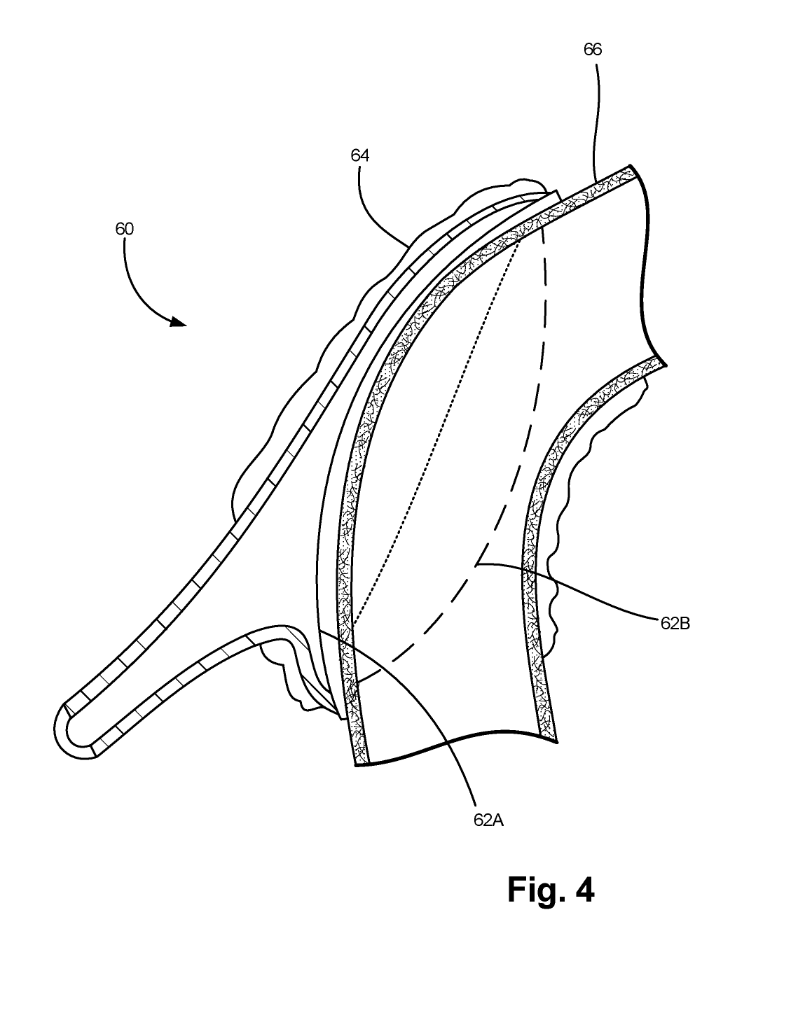

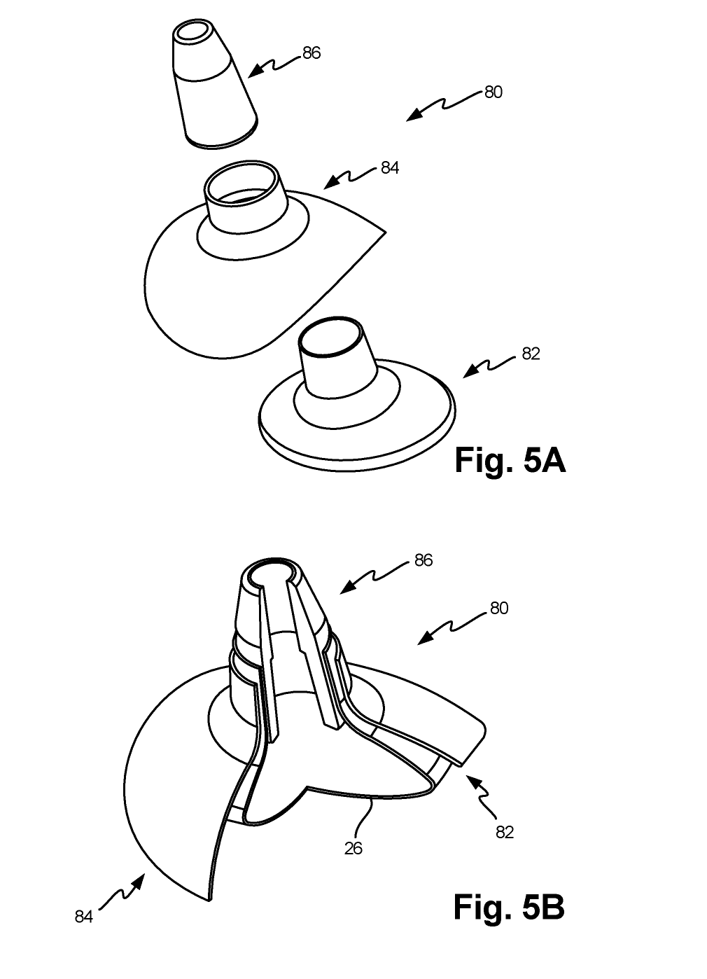

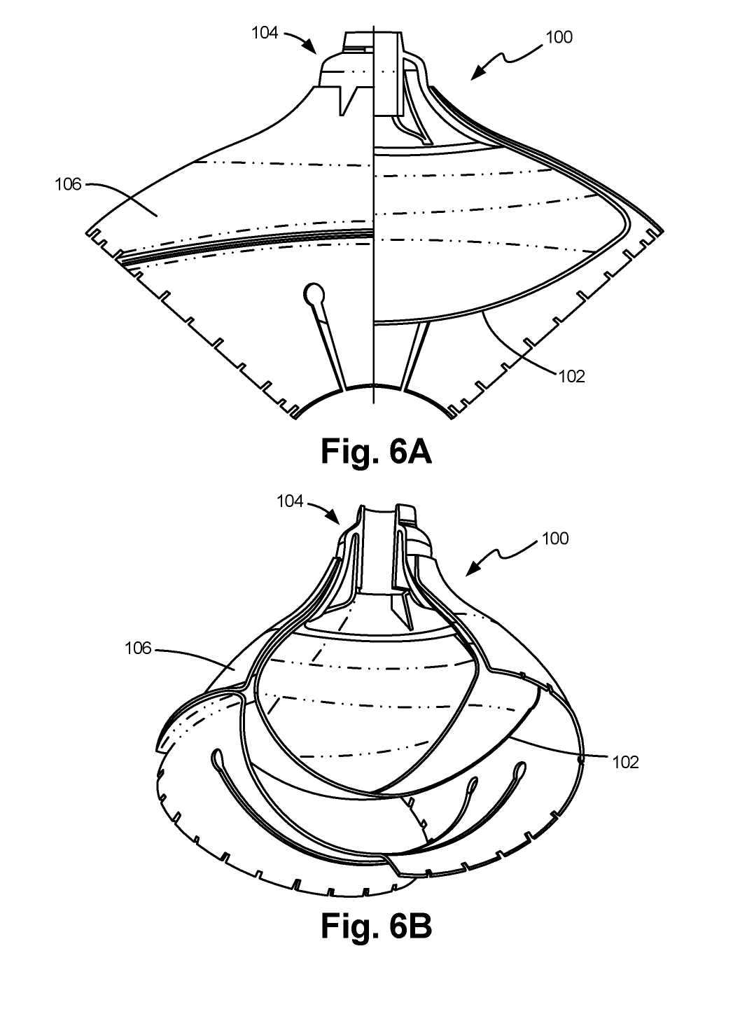

As will be described in further detail below, the actuator 12 can be any type of actuator that can be used to compress the pulmonary artery 22. For example, in certain implementations, the actuator 12 is similar to the actuator 40 depicted in FIGS. 3A and 3B having coils 42, 44 made of an electro-active polymer. Alternatively, the actuator 12 is a device with an inflatable membrane similar to the actuator 60 depicted in FIG. 4, an actuator 12 with an inflatable balloon similar to the actuators 80, 100 depicted in FIGS. 5A-5B and 6A-6B, respectively, or any other such actuator.

In various embodiments, the actuator 12 can be any type of actuator, including fluidically (including hydraulically), electrically, or magnetically driven actuators. For example, the actuator 12 can be driven by a fluid such as a liquid or a gas. With respect to electricity-driven implementations, the actuator 12 can, for example, be driven by an electric motor or by activation of electro-active polymers (by passing electricity through the polymers). In the magnetically-driven embodiments, the actuator 12 can be driven by magnetization of a conductive fixture (thereby moving it against the pulmonary artery).

In further alternatives, the actuator 12 could be a mechanical actuator such as a piston or other mechanical device, an actuator with an electro-active polymer, or an actuator with a polymer with conductive coils embedded in them.

As shown in FIG. 1, the system also has a controller 14 that is coupled to the actuator 12 via a percutaneous interface line 16. In those embodiments in which the actuator 12 is hydraulically driven, the controller 14 can have a pump (not shown) that drives the actuator 12. In such an implementation, the interface line 16 has a fluid line that allows for transfer of the fluid pressure to the actuator 12. Alternatively, the pump can be implanted in the patient and operably coupled to the actuator 12 such that the controller 14 is operably coupled to the pump via the interface line 16. In those embodiments in which the actuator 12 is magnetically or electrically driven, the controller 14 can have an electrical power source that powers the actuator 12 such that the interface line 16 includes an electrical cable that transfers the electricity from the electrical power source associated with the controller 14 to the actuator 12.

In some embodiments, the controller 14 has a transceiver that allows the controller 14 to communicate wirelessly with the actuator 12, any implanted pump (not shown), or any other component of the system. In further alternatives, the controller 14 is implanted in the chest cavity.

According to certain implementations, the controller 14 has a processor with memory (or a separate memory component) that stores the operational logic required to control the controller 14 and actuator 12. As such, the controller 14 can, according to some embodiments, be configured to control the actuator to provide counterpulsation of the pulmonary artery.

Whether it is electrical, fluidic, magnetic, or otherwise, the motive component in certain embodiments is designed so that in the event of failure, it automatically goes into “off” with the actuator in its non-compressed position so that the pulmonary artery is not compressed, thus minimizing risk to the patient.

Further, in various implementations, the motive component can include or be associated with a component for detecting speed and completeness of actuator compression and retraction, measuring the amount of pressure applied to the artery during compression, and/or measuring arterial blood pressure or flow in the pulmonary artery.

The power source for the system can be an internal and/or external battery (which can be in or associated with the controller), or TET (transcutaneous electronic transfer). One example of an internal battery would be a battery similar to that used in a pacemaker, CRT-D device, or any other similar electrical stimulation device.

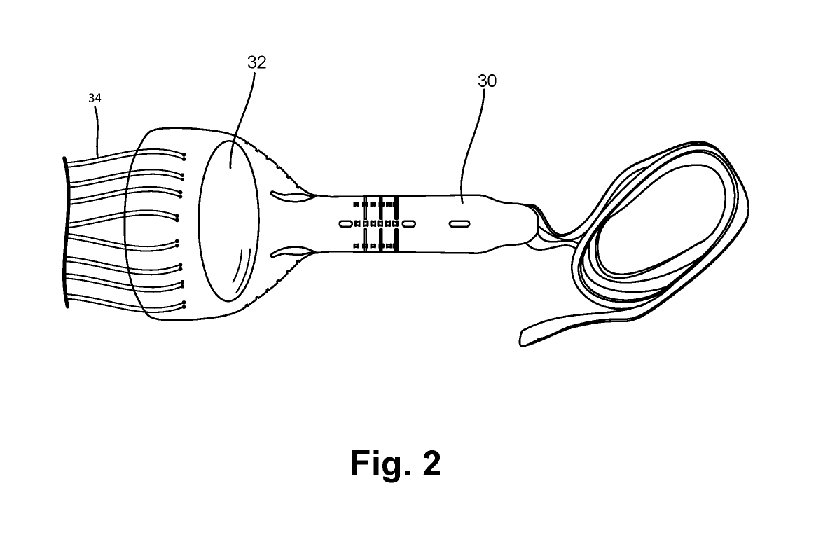

The actuator 12 can be positioned against or around the pulmonary artery 22 by any known device or means. For example, in one system embodiment as depicted in FIG. 2, a wrap 30 is provided that can be positioned around the actuator 32 and the pulmonary artery 22 and affixed to itself via the sutures 34, thereby retaining the actuator 32 against or adjacent to the pulmonary artery 22 such that inflation of the actuator 32 causes compression of the artery 22.

It is further understood that any of the actuator embodiments disclosed or contemplated herein can be attached to or positioned against the pulmonary artery 22 using any number of devices or methods. For example, the actuator can be attached or positioned against the artery 22 via suturing, gluing, suturing tabs, Velcro, magnets, an interference fit, apertures allowing in-growth of tissue, surface portions adapted to promote tissue growth into or onto the actuator so as to hold the device in position relative to the pulmonary artery, or any other known attachment or retention device or component.

FIGS. 3A and 3B depict a further embodiment of an actuator 40, in which the actuator 40 has coils 42, 44 made of an electro-active polymer. The coils 42, 44 of the actuator 40 are positioned on opposite sides of the pulmonary artery 48 such that expansion of the coils 42, 44 causes compression of the artery 48. The actuator 40 is electrically coupled to a controller (not shown) via an electrical cable 46 or other type of electrical connection component. In use, electricity is applied to the coils 42, 44 via the electrical cable 46, thereby activating the electro-active polymer in the coils 42, 44, thereby causing the coils 42, 44 to expand and thereby compress the pulmonary artery 48.

Another embodiment of an actuator 60 is shown in FIG. 4, which depicts an actuator 60 with an inflatable membrane 62 that can be positioned against the pulmonary artery 66 and retained in place with a wrap 64. The line identified as 62A is the membrane 62 in its uninflated position, while 62B show the membrane 62 in its inflated state. The actuator 60 is described in further detail in U.S. Pat. No. 7,347,811, which is hereby incorporated herein by reference in its entirety.

A further embodiment of an actuator 80 is shown in FIGS. 5A and 5B, which depict an actuator 80 having a flexible, inflatable balloon 82 that can be positioned against the pulmonary artery (not shown). The actuator 80 also has a substantially inelastic shroud 84 and a bushing 86. The actuator is described in further detail in U.S. Pat. No. 7,955,248, which is hereby incorporated herein by reference in its entirety.

Yet another embodiment of an actuator 100 is shown in FIGS. 6A and 6B, which depict an actuator 100 having a flexible, inflatable balloon 102 that can be positioned against the pulmonary artery (not shown). The actuator 100 also has a bushing 104 and a flexible, relatively inelastic wrap 106. This actuator is also described in further detail in U.S. Pat. No. 7,955,248, which is mentioned and incorporated herein above.

In one implementation, the system and device embodiments disclosed and contemplated herein can lead to an increase pulmonary compliance associated with a reduction in resistance, thereby improving the pulmonary time constant due to the changes in resistance and compliance being reciprocal. The increase in pulmonary compliance and reduction in resistance can lead to a reduction in the work of breathing, alleviating the sensation of dyspnea, and minimizing the swings in pleural pressure helping to unload the left and right heart.

According to another embodiment, the various implementations of actuator devices for physically stressing the pulmonary artery can improve filling of the left ventricle by causing an increased E wave and reduced A-wave, improving diastolic function of the left ventricle, and reducing filling/wedge pressures. More specifically, positioning an actuator adjacent to the pulmonary artery and causing inflation of that actuator that is timed to diastole (dicrotic notch) or atrial systole (P wave) will create a forward compression wave leading to increased forward flow into the left ventricle and unloading of the left atrium and right ventricle. In addition to the forward compression wave, rapid deflation of the actuator can further enhance expansion waves generated by the right ventricle, thereby leading to further reduction in right ventricle afterload. This right ventricle afterload reduction also helps to further improve left ventricle filling by reducing the stress in the septum on the right side, thereby allowing it to shift leftward toward to the right ventricle during left ventricle filling.

One advantage of the device and system embodiments disclosed herein is that the risk of limb ischemia associated with blood-contacting systems is avoided because there is no blood contact with the device whatsoever.

According to one embodiment, the actuator is adapted to squeeze from about 10 mL to about 25 ml of blood from the pulmonary artery in each compression cycle.

In further implementations, any of the various system and device embodiments disclosed or contemplated herein for compressing or otherwise externally, physically stressing the pulmonary artery can be combined with any known system or device for compressing or otherwise deforming the ascending aorta, thereby resulting in various systems, methods, and devices for compressing both the pulmonary artery and the ascending aorta. For example, any of the pulmonary artery actuator devices disclosed or contemplated herein can be combined with the aortic compression devices and systems disclosed in any of U.S. Pat. Nos. 8,002,691, 8,425,397, 8,591,394, and/or 8,702,583, or U.S. Published Applications 2014/0296616, 2014/0051909, 2014/0148639, 2013/0310629, 2014/0094645, and/or 2014/0257019, all of which are hereby incorporated herein by reference in their entireties.

Although certain embodiments have been described herein, persons skilled in the art will recognize that changes may be made in form and detail without departing from the spirit and scope of the invention.

What is claimed is:

1. A heart assist system comprising:
(a) an arterial compression device configured to be positioned adjacent to a pulmonary artery of a patient, wherein the arterial compression device is configured to compress the pulmonary artery, whereby the arterial compression device is configured to cause the pulmonary artery to release nitric oxide;
(b) a pump in fluid communication with the arterial compression device, wherein the pump is configured to pump a fluid to the arterial compression device so as to actuate the arterial compression device; and
(c) a power source operably coupled with the pump, the power source comprising a battery or a transcutaneous electronic transfer device.

2. A heart assist system comprising:
(a) an arterial compression device configured to be positioned adjacent to a pulmonary artery of a patient, wherein the arterial compression device is configured to compress the pulmonary artery, whereby the arterial compression device is configured to cause improved filling of a left ventricle of the patient;
(b) a pump in fluid communication with the arterial compression device, wherein the pump is configured to pump a fluid to the arterial compression device so as to actuate the arterial compression device; and
(c) a power source operably coupled with the pump, the power source comprising a battery or a transcutaneous electronic transfer device.

3. The heart assist system of claim 2, wherein the arterial compression device is configured to cause improved filling of the left ventricle by increasing forward flow into the left ventricle and reducing afterload in a right ventricle.

4. A method of treating pulmonary arterial hypertension, the method comprising:
positioning an arterial compression device adjacent to a pulmonary artery of a patient; and
compressing the pulmonary artery with the arterial compression device, whereby the arterial compression device is configured to cause the pulmonary artery to release nitric oxide.

5. A method of treating pulmonary arterial hypertension, the method comprising:
positioning an arterial compression device adjacent to a pulmonary artery of a patient; and
causing the pulmonary artery to release nitric oxide by compressing the pulmonary artery with the arterial compression device.

6. A method of treating pulmonary arterial hypertension, the method comprising:
positioning an arterial compression device adjacent to a pulmonary artery of a patient; and
compressing the pulmonary artery with the arterial compression device, whereby the arterial compression device is configured to cause improved filling of a left ventricle of the patient.

7. The method of claim 6, wherein the arterial compression device is configured to cause improved filling of the left ventricle by increasing forward flow into the left ventricle and reducing afterload in a right ventricle.

8. A method of treating pulmonary arterial hypertension, the method comprising:
positioning an arterial compression device adjacent to a pulmonary artery of a patient; and
causing improved filling of a left ventricle of the patient by compressing the pulmonary artery with the arterial compression device.
